(19) United States
(12) Patent Application Publication
(10) Pub. No.: US 2021/0161883 A1
(43) Pub. Date: Jun. 3, 2021

(54) EZH2 INHIBITOR-INDUCED GENE EXPRESSION

(71) Applicant: Constellation Pharmaceuticals, Inc., Cambridge, MA (US)

(72) Inventors: William D. Bradley, Winchester, MA (US); Barbara M. Bryant, Cambridge, MA (US); Patrick Trojer, Reading, MA (US)

(21) Appl. No.: 16/629,641

(22) PCT Filed: Jul. 10, 2018
(86) PCT No.: PCT/US2018/041404

§ 371 Date: Jan. 9, 2020

Related U.S. Application Data
(60) Provisional application No. 62/530,392, filed on Jul. 10, 2017.

(51) Int. Cl. A61K 31/4545; A61P 35/00

(52) U.S. Cl. CPC A61K 31/4545; A61P 35/00

(57) ABSTRACT

Provided herein are methods of treating subjects characterized as having a gene signature that is indicative of EZH2-inhibitor response, methods for determining said gene signature, and methods of determining which subjects may be responsive to cancer treatments based on said gene signature.

### RELATED APPLICATIONS

This application claims the benefit of priority to U.S. Provisional Application No. 62/530,392, filed Jul. 10, 2017, the entire contents of which are incorporated herein by reference.

### BACKGROUND

Drug efficacy depends on the extent to which a drug binds its respective target. Adverse effects of drugs typically arise from excessive or off-target binding. Thus, a critical step in the pharmacological evaluation of drugs is the ability to determine if the drug is engaging with its respective target. EZH2 (Enhancer of Zeste Homolog 2) is a histone lysine methyltransferase that has been implicated in the pathogenesis of both hematologic and non-hematologic malignancies. Therapeutics that specifically target EZH2 have been developed and are presently in clinical trials for treating a variety of cancers.

While the development of new EZH2 inhibitors remains a focus for some, the identification of gene expression profiles as they relate to target engagement remains an unmet challenge. The ability to correlate the level of EZH2 engagement with pre-clinical therapeutic responses provides the practitioner with an alternative pharmacodynamics marker for assessing if engagement and/or proper dosing of the particular EZH2 inhibitor has been met. In some instances, it may also provide means for determining whether the administration of a particular EZH2 inhibitor is likely to elicit a therapeutic response, thus assisting in the determination of proper patient selection.

### SUMMARY

Here, it has been found that administration of the EZH2 inhibitor (R)—N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide, herein Compound 1, causes a change in the expression level of one or more of the 46 genes in the disclosed gene signature. The change in expression levels of one or more of the identified genes was found to be controlled by EZH2 inhibition and was sensitive enough to distinguish between Compound 1-treated and untreated lymphoma cell models in vitro and in vivo. See e.g., FIGS. 1A and B, illustrating the detection of Compound 1-mediated gene expression changes in Karpas-422 GCB-DLBCL cells. The structure of Compound 1 is shown below.

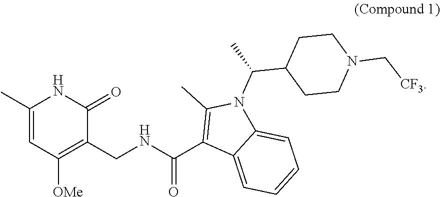 [SMILES: COc1cc(C)[nH]c(=O)c1CNC(=O)c1c(C)n([C@H](C)C2CCN(CC(F)(F)F)CC2)c2ccccc12]

This EZH2-controlled gene expression can be used to determine if an EZH2 inhibitor, such as compound 1, is engaging with the target, i.e., EZH2. See e.g., FIG. 4, which shows significant changes in signature gene expression in tumor samples derived from Karpas-422 xenograft bearing mice treated with various dosages of Compound 1. From this, one can assess the level of target engagement and determine whether there is a sufficient target engagement to result in a therapeutic response.

Disclosed herein, therefore, are methods of treating cancer in a subject characterized as having altered gene expression of one or more genes selected from the disclosed gene signature with an EZH2 inhibitor such as Compound 1.

Also disclosed are methods for determining altered gene expression and methods for adjusting the amount of an administered EZH2 inhibitor based upon target engagement determined by a change in the expression level of one or more genes selected from the disclosed gene signature.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A and B illustrate the detection of Compound 1-mediated gene expression changes in Karpas-422 GCB-DLBCL cells where (A) represents Karpas-422 cells treated with DMSO or 1.5 μM of various EZH2 inhibitors and (B) represents a heatmap representing 604 genes that were significantly altered (log 2 fold change (FC) >0.8, p<0.05) by all compounds in comparison to the DMSO-treated controls.

FIGS. 2A, B, and C illustrate the identification of an EZH2-controlled gene expression program in DLBCL, where (A) is a summary of the inclusion criteria that led to the identification of 339 upregulated and 213 downregulated genes in response to EZH2 inhibitor treatment across 14 lymphoma cell lines; (B) represents a heatmap representation of an EZH2-controlled 552 gene signature with upregulated and downregulated genes shown in red and blue, respectively; and (C) represents a gene signature in 7 DLBCL cell lines.

FIG. 3 illustrates the measuring of the EZH2-controlled gene signature in Karpas-422 cell line and tumor samples.

FIGS. 4A and B illustrate a comparison of Compound 1-mediated H3K27me3 level and gene expression changes in phenotypically responsive Karpas-422 xenografts where (A) represents tumor samples (n=4) from a Karpas-422 mouse xenograft experiment in which mice were dosed twice daily, orally with vehicle, 100 or 200 mg/kg of Compound 1 for 4, 7, or 14 days, and were analyzed by ELISA assays to determine H3K27me3 levels; and (B) is the same tumor samples described in (A) (n=4), but were analyzed by a multiplexed QuantiGene® assay for the transcript levels of 50 genes, 4 of which were housekeeping.

FIGS. 5A and B illustrate a xenograft model that does not phenotypically respond, but does show target engagement, where A represents tumor volume from a RL mouse xenograft experiment in which mice were dosed twice daily, subcutaneously with vehicle or 200 mg/kg of Compound 2 and B represents tumor samples from the experiment described in (A) that were analyzed by a multiplexed QuantiGene® assay for the transcript levels of 50 genes, 4 of which were housekeeping.

### DETAILED DESCRIPTION

Disclosed herein are methods of treating cancer in a subject, comprising a) administering to the subject an initial dosage amount of an EZH2 inhibitor; b) determining the change in the expression level relative to a baseline level of two or more genes (e.g., at least five genes) in the subject selected from TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1; and c) adjusting the initial dosage amount of the EZH2 inhibitor being administered to the subject to an adjusted dosage amount, such that the adjusted dosage amount results in a statistically significant change relative to the baseline level of the selected genes. In one aspect, the disclosed methods further comprise the step of calculating a target engagement gene score from the expression level changes of the at least five genes.

In another aspect, the present methods comprise treating cancer in a subject, comprising a) administering to the subject an initial dosage amount of an EZH2 inhibitor; b) determining the level of change in the expression level from a baseline level of two or more genes (e.g., at least five genes) in the subject selected from TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1; and c) if the change in the expression level of at least five genes is not statistically significant relative to the baseline level of the selected genes, adjusting the initial dosage amount of the EZH2 inhibitor being administered to the subject to an adjusted dosage amount, such that the adjusted dosage amount results in a statistically significant change in the expression level relative to the baseline level of the selected genes; or d) if the change in the expression level of the at least five genes is statistically significant relative to the baseline level of the selected genes, continuing to administer to the subject the initial dosage amount of the EZH2 inhibitor. Steps b) and c) may be repeated, if necessary, until the dosage amount results in a statistically significant change in the expression level relative to the baseline level of the selected genes.

In the methods described herein, a sample such as a biopsy may be taken from the subject's cancer prior to treatment in order to determine the expression level of the selected genes.

The amount of time which transpires between administration of an effective amount of an EZH2 inhibitor and determining the change in expression is at least the amount of time required for the EZH2 inhibitor elicit a statistically significant change in expression in the selected genes. In one aspect, the time required for the EZH2 inhibitor to elicit a statistically significant change in expression levels is one day, two days, three days, four days, five days, six days, seven days, up to 1-month or greater after administration of the EZH2 inhibitor. In one aspect, the time required for the EZH2 inhibitor to elicit a change in expression levels is at least 28 days after administration of the EZH2 inhibitor

In one aspect, gene expression can be determined by qPCR, e.g., cells can be harvested and total RNA isolated using commercially available methods. Reverse transcription can then be carried out using commercially available methods. Quantitative PCR can then be performed using commercially available methods. Target gene mRNA levels can then be assessed using commercially available methods e.g., gene-specific probes. This can be compared with an internal control.

As used herein, “baseline level” as in “a statistically significant change from the baseline level of the selected genes” means the expression level of one or more of the disclosed genes when the concentration of the EZH2 inhibitor in the blood of the subject is below the level of detection. Thus, the baseline level includes subjects who have never been treated with an EZH2 inhibitor or subjects who have been previously treated with an EZH2 inhibitor, but where detectable amounts of the EZH2 inhibitor are no longer present in the subject. In one aspect, “baseline level” refers to subjects who have never been treated with an EZH2 inhibitor.

The term “initial dose” or “initial dosage amount” is the amount of an EZH2 inhibitor, which when administered to a subject having a cancer, is expected to elicit a response of the subject's cancer such as eliciting a statistically significant change in the expression of one or more genes (e.g., at least five genes) of the disclosed gene signature. The initial dose may be selected from the experience of the attending physician or from the recommended amount based on clinical trials. The exact amount required can vary from subject to subject, depending on the species, age, and general condition of the subject, the severity of disease (or underlying genetic defect) that is being treated, the particular compound used, its mode of administration, and the like. Whether the subject will continue to receive the initial dose is determined by the target engagement of the EZH2 inhibitor. “Target engagement” refers to the extent to which the EZH2 inhibitor is inhibiting EZH2 within the tumor and the extent to which the inhibition of EZH2 is causing changes in gene expression for those genes whose expression is affected by EZH2 inhibition. Target engagement is measured by determining the change in expression level from the baseline level of the gene signature disclosed herein. If there is sufficient target engagement, administration of the initial dosage amount is continued.

“Adjusted dosage amount” or “adjusted dose” of the EZH2 inhibitor is the quantity which results in a statistically significant change in gene expression is generally continually administered until treatment is terminated. In one aspect, the quantity of EZH2 inhibitor that is administered to the subject is increased above the adjusted dosage amount (or above an initial dosage amount that results in a statically significant change in gene expression relative to the baseline level), provided that the increased dosage amount is tolerated by the subject, i.e., is not toxic and does not cause unacceptable side effects.

In instances where there is sufficient target engagement, the dose being administered to the subject is such that a statistically significant change in the expression level relative to a baseline level of at least five genes in the disclosed gene sequence is realized.

In instances where there is insufficient target engagement, the dose being administered to the subject is modified until the adjusted dosage amount is achieved. For example, if after administration of the initial dosage amount a statistically significant change in the expression level relative to a baseline level of at least five genes in the disclosed gene signature is not achieved, the amount of EZH2 inhibitor is increased. The change in expression level from the baseline level of the gene signature is again determined. The process of adjustment, administration of the EZH2 inhibitor, and assessment is continued until the adjusted dosage amount is reached, i.e., there is there is a statistically significant change in the expression level relative to a baseline level of at least five genes from the disclosed gene signature.

Changes in gene expression that occur between any two treatment conditions (e.g., baseline and after administration of an initial dosage amount) are statistically significant when the change in gene expression or the mean change in gene expression differ sufficiently outside of the technical error threshold of a particular assay platform. The technical error threshold is dependent on the assay platform used to detect gene expression levels, and will vary from platform to platform. Specifically, “statistically significant”, as used herein, means a change in gene expression or a mean change in gene expression after treatment that is at least 1.96 times the standard deviation greater than the corresponding value at baseline“. Alternatively, the change in gene expression or the mean change in gene expression after treatment is at least 1.96, 2.33, 2.58, 2.81, 3.09, 3.30 times the standard deviation greater than the corresponding value at baseline”. Using a Z-test, these values correspond to 95%, 98%, 99%, 99.5%, 99.8%, and 99.9% confidence intervals, respectively.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The terms “treatment,” “treat,” and “treating” refer to reversing, alleviating, or inhibiting the progress of a cancer, or one or more symptoms thereof, as described herein. In some embodiments, treatment may be administered after one or more symptoms have developed, i.e., therapeutic treatment. Treatment may also be continued after symptoms have resolved, for example to reduce the likelihood or delay their recurrence.

In each of the methods described herein, at least five genes from the disclosed gene signature can be used for characterization or analysis. For example, in each of the preceding embodiments, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-one, at least thirty-two, at least thirty-three, at least thirty-four, at least thirty-five, at least thirty-six, at least thirty-seven, at least thirty-eight, at least thirty-nine, at least forty, at least forty-one, at least forty-two, at least forty-three, at least forty-four, at least forty-five, or all forty-six genes in the pared gene signature.

As used herein, “gene signature” or “disclosed gene signature” refers to the genes TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1.

As used herein, “altered expression level”, “change in expression level”, or “change in the level of expression” of one or more genes disclosed herein mean that there is either a decrease or increase in the level of expression of the five or more genes from baseline following the administration of an EZH2 inhibitor. When used in the context of a change in expression level, downregulation or downregulated means there is a decrease in the level of expression of the one or more genes from baseline following the administration of an EZH2 inhibitor. When used in the context of a change in expression level, upregulation or upregulated means there is an increase in the level of expression of the one or more genes from baseline following the administration of an EZH2 inhibitor. In one aspect, a change in the expression level of the five or more genes means that there is a decrease or increase in the level of expression of the five or more genes such that the level of target engagement from the EZH2 inhibitor is sufficient enough to enable the likelihood of producing a therapeutic response. In some aspects, a change in gene expression or the mean change in gene expression after treatment means that there is either a decrease or increase in the level of expression of the five or more genes such that there is a change of at least 1.96, 2.33, 2.58, 2.81, 3.09, 3.30 times the standard deviation greater than the corresponding value at baseline following the administration of an EZH2 inhibitor. In one aspect, “change of expression” for TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, and ABAT means upregulation (i.e., increased expression), and “change of expression” for HAUS8, CENPQ, RRM1, ATAD2, PBK, RAD51, RAD51AP1, CKS1B, and MND1 means downregulation (i.e., decreased expression). Alternatively, the expression level of at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, at least twenty-two, at least twenty-three, at least twenty-four, at least twenty-five, at least twenty-six, at least twenty-seven, at least twenty-eight, at least twenty-nine, at least thirty, at least thirty-one, at least thirty-two, at least thirty-three, at least thirty-four, at least thirty-five, and at least thirty-six of TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, and ABAT is characterized by upregulation (i.e., increased). In another alternative, the expression level of TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, and ABAT is characterized by upregulation (i.e., increased).

In one aspect, the expression level of at least one of HAUS8, CENPQ, RRM1, ATAD2, PBK, RAD51, RAD51AP1, CKS1B, and MND1 is downregulated (i.e., decreased). Alternatively, the expression level of at least five, at least six, at least seven, and at least eight of HAUS8, CENPQ, RRM1, ATAD2, PBK, RAD51, RAD51AP1, CKS1B, and MND1 is characterized by downregulation (i.e., decreased). In another alternative, the expression level of HAUS8, CENPQ, RRM1, ATAD2, PBK, RAD51, RAD51AP1, CKS1B, and MND1 is characterized by downregulation (i.e., decreased).

In one aspect, the change in gene expression for TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, and ABAT is upregulation, and the change in gene expression for HAUS8, CENPQ, RRM1, ATAD2, PBK, RAD51, RAD51AP1, CKS1B, and MND1 is downregulation. In another aspect, each gene in the disclosed gene signature is upregulated or downregulated, wherein TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, and ABAT is upregulated and HAUS8, CENPQ, RRM1, ATAD2, PBK, RAD51, RAD51AP1, CKS1B, and MND1 is downregulated.

In one aspect, in instances where a statistically significant change in the expression level relative to the baseline level of the selected genes cannot be achieved, cancer therapies other than an EZH2 inhibitor may be administered to the subject. These therapies include, but are not limited to, surgery, radiation therapy, immunotherapy, endocrine therapy, gene therapy and administration of an anti-cancer agent other than an EZH2 inhibitor.

In one aspect, in instances where a statistically significant change in the expression level relative to the baseline level of the selected genes is achieved, cancer therapies comprising combinations of EZH2 inhibitors may be administered to the subject may be administered to the subject. These therapies include, but are not limited to, surgery, radiation therapy, immunotherapy, endocrine therapy, gene therapy and administration of an anti-cancer agent other than an EZH2 inhibitor.

An endocrine therapy is a treatment that adds, blocks or removes hormones. For example, chemotherapeutic agents that can block the production or activity of estrogen have been used for treating breast cancer. In addition, hormonal stimulation of the immune system has been used to treat specific cancers, such as renal cell carcinoma and melanoma. In one embodiment, the endocrine therapy comprises administration of natural hormones, synthetic hormones or other synthetic molecules that may block or increase the production or activity of the body's natural hormones. In another embodiment, the endocrine therapy includes removal of a gland that makes a certain hormone.

A gene therapy is the insertion of genes into a subject's cell and biological tissues to treat diseases, such as cancer. Exemplary gene therapy includes, but is not limited to, a germ line gene therapy and a somatic gene therapy.

Immunotherapy (also called biological response modifier therapy, biologic therapy, biotherapy, immune therapy, or biological therapy) is treatment that uses parts of the immune system to fight disease. Immunotherapy can help the immune system recognize cancer cells, or enhance a response against cancer cells. Immunotherapies include active and passive immunotherapies. Active immunotherapies stimulate the body's own immune system while passive immunotherapies generally use immune system components created outside of the body. Examples of active immunotherapies include, but are not limited to vaccines including cancer vaccines, tumor cell vaccines (autologous or allogeneic), dendritic cell vaccines, antigen vaccines, anti-idiotype vaccines, DNA vaccines, viral vaccines, or Tumor-Infiltrating Lymphocyte (TIL) Vaccine with Interleukin-2 (IL-2) or Lymphokine-Activated Killer (LAK) Cell Therapy. Examples of passive immunotherapies include but are not limited to monoclonal antibodies and targeted therapies containing toxins. Monoclonal antibodies include naked antibodies and conjugated monoclonal antibodies (also called tagged, labeled, or loaded antibodies). Naked monoclonal antibodies do not have a drug or radioactive material attached whereas conjugated monoclonal antibodies are joined to, for example, a chemotherapy drug (chemolabeled), a radioactive particle (radiolabeled), or a toxin (immunotoxin). Examples of these naked monoclonal antibody drugs include, but are not limited to Rituximab (Rituxan), an antibody against the CD20 antigen used to treat, for example, B cell non-Hodgkin lymphoma; Trastuzumab (Herceptin), an antibody against the HER2 protein used to treat, for example, advanced breast cancer; Alemtuzumab (Campath), an antibody against the CD52 antigen used to treat, for example, B cell chronic lymphocytic leukemia (B-CLL); Cetuximab (Erbitux), an antibody against the EGFR protein used, for example, in combination with irinotecan to treat, for example, advanced colorectal cancer and head and neck cancers; and Bevacizumab (Avastin) which is an antiangiogenesis therapy that works against the VEGF protein and is used, for example, in combination with chemotherapy to treat, for example, metastatic colorectal cancer. Examples of the conjugated monoclonal antibodies include, but are not limited to Radiolabeled antibody Ibritumomab tiuxetan (Zevalin) which delivers radioactivity directly to cancerous B lymphocytes and is used to treat, for example, B cell non-Hodgkin lymphoma; radiolabeled antibody Tositumomab (Bexxar) which is used to treat, for example, certain types of non-Hodgkin lymphoma; and immunotoxin Gemtuzumab ozogamicin (Mylotarg) which contains calicheamicin and is used to treat, for example, acute myelogenous leukemia (AML). BL22 is a conjugated monoclonal antibody for treating, for example, hairy cell leukemia, immunotoxins for treating, for example, leukemias, lymphomas, and brain tumors, and radiolabeled antibodies such as OncoScint for example, for colorectal and ovarian cancers and ProstaScint for example, for prostate cancers.

Immunotherapies that can be used in the present teachings include adjuvant immunotherapies. Examples include cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guérin (BCG); Keyhole limpet hemocyanin (KLH); Incomplete Freund's adjuvant (IFA); QS-21; DETOX; Levamisole; and Dinitrophenyl (DNP), and combinations thereof, such as, for example, combinations of, interleukins, for example, IL-2 with other cytokines, such as IFN-alpha.

In one aspect, cancer therapies other than an EZH2 inhibitor are compounds, which when administered in a therapeutically effective amount to a subject with cancer, can achieve, partially or substantially, one or more of the following: arresting the growth, reducing the extent of a cancer (e.g., reducing size of a tumor), inhibiting the growth rate of a cancer, and ameliorating or improving a clinical symptom or indicator associated with a cancer (such as tissue or serum components), or increasing longevity of the subject.

The anti-cancer agent suitable for use in the methods described herein include anti-cancer agents that have been approved for the treatment of cancer. In one aspect, the anti-cancer agent includes, but is not limited to, a targeted antibody, an angiogenisis inhibitor, an alkylating agent, an antimetabolite, a vinca alkaloid, a taxane, a podophyllotoxin, a topoisomerase inhibitor, a hormonal antineoplastic agent and other antineoplastic agents.

Examples of alkylating agents useful in the methods of the present teachings include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites useful in the methods of the present teachings include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of plant alkaloids and terpenoids or derivatives thereof include, but are not limited to, vinca alkaloids (e.g., vincristine, vinblastine, vinorelbine, vindesine), podophyllotoxin, and taxanes (e.g., paclitaxel, docetaxel). Examples of a topoisomerase inhibitor includes, but is not limited to, irinotecan, topotecan, amsacrine, etoposide, etoposide phosphate and teniposide. Examples of antineoplastic agents include, but are not limited to, actinomycin, anthracyclines (e.g., doxorubicin, daunorubicin, valrubicin, idarubicin, epirubicin), bleomycin, plicamycin and mitomycin.

In one aspect, the anti-cancer agents that can be used in the present teachings include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofo sine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; tenipo side; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents/drugs that can be used in the present teachings include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

In one aspect, cancer therapies are anti-cancer agents suitable for treating leukemias. Exemplary treatments include, but are not limited to, Abitrexate® (Methotrexate), Arranon® (Nelarabine), Asparaginase Erwinia chrysanthemi, Blinatumomab, Blincyto® (Blinatumomab), Cerubidine® (Daunorubicin Hydrochloride), Clafen® (Cyclophosphamide), Clofarabine®, Clofarex® (Clofarabine), Clolar® (Clofarabine), Cyclophosphamide, Cytarabine, Cytosar-U® (Cytarabine), Cytoxan® (Cyclophosphamide), Dasatinib, Daunorubicin Hydrochloride, Doxorubicin Hydrochloride, Erwinaze® (Asparaginase Erwinia Chrysanthemi), Folex® (Methotrexate), Folex PFS® (Methotrexate), Gleevec® (Imatinib Mesylate), Iclusig® (Ponatinib Hydrochloride), Imatinib Mesylate, Marqibo® (Vincristine Sulfate Liposome), Mercaptopurine, Methotrexate, Methotrexate LPF® (Methorexate), Mexate® (Methotrexate), Mexate-AQ® (Methotrexate), Nelarabine, Neosar® (Cyclophosphamide), Oncaspar® (Pegaspargase), Pegaspargase, Ponatinib Hydrochloride, Prednisone, Purinethol® (Mercaptopurine), Purixan® (Mercaptopurine), Rubidomycin® (Daunorubicin Hydrochloride), Spryce®l (Dasatinib), Tarabine PFS® (Cytarabine), Vincasar PFS® (Vincristine Sulfate), Vincristine Sulfate, Vincristine Sulfate Liposome, Hyper-CVAD, Arsenic Trioxide, Idamycin (Idarubicin Hydrochloride), Idarubicin Hydrochloride, Mitoxantrone Hydrochloride, Tabloid (Thioguanine), Thioguanine, Trisenox® (Arsenic Trioxide), Alemtuzumab, Ambochlorin® (Chlorambucil), Arzerra® (Ofatumumab), Bendamustine Hydrochloride, Campath® (Alemtuzumab), Chlorambucil, Fludara® (Fludarabine Phosphate), Fludarabine Phosphate, Gazyva® (Obinutuzumab), Ibrutinib, Idelalisib, Imbruvica® (Ibrutinib), Leukeran® (Chlorambucil), Linfolizin® (Chlorambucil), Mechlorethamine Hydrochloride, Mustargen® (Mechlorethamine Hydrochloride), Obinutuzumab, Ofatumumab, Rituxan® (Rituximab), Rituximab, Treanda® (Bendamustine Hydrochloride), Venclexta® (Venetoclax), Venetoclax, Zydelig® (Idelalisib), chlorambucil-prednisone, CVP, Bosulif (Bosutinib), Bosutinib, Busulfan, Busulfex (Busulfan), Hydrea® (Hydroxyurea), Hydroxyurea, Mechlorethamine Hydrochloride, Myleran® (Busulfan), Neosar (Cyclophosphamide), Nilotinib, Omacetaxine Mepesuccinate, Synribo® (Omacetaxine Mepesuccinate), and Tasigna® (Nilotinib).

EZH2 inhibitors described herein include e.g., small molecules that are capable of inhibiting EZH2 activity. Inhibition can be measured in vitro, in vivo, or from a combination thereof. In one aspect, the EZH2 inhibitors in the methods described herein include, but are not limited to,

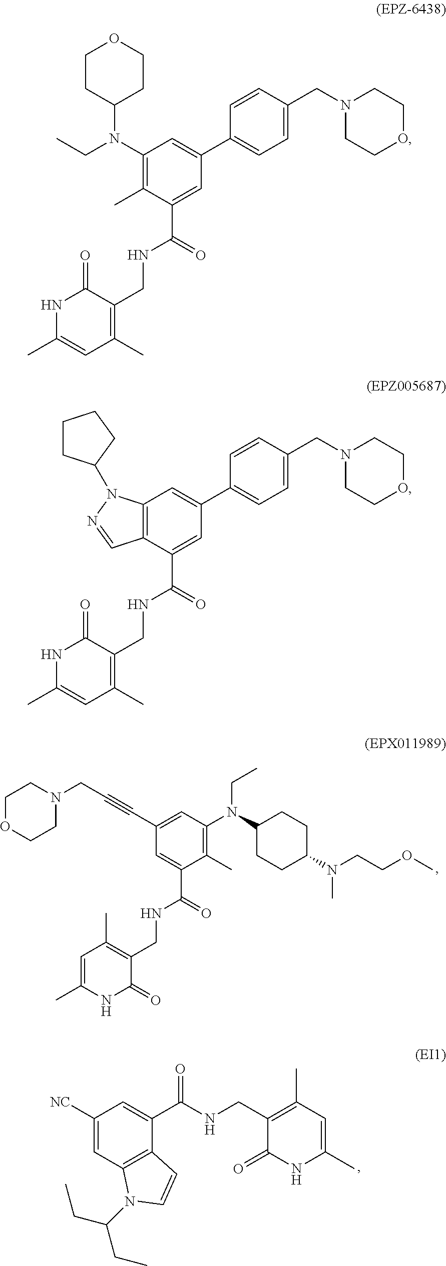

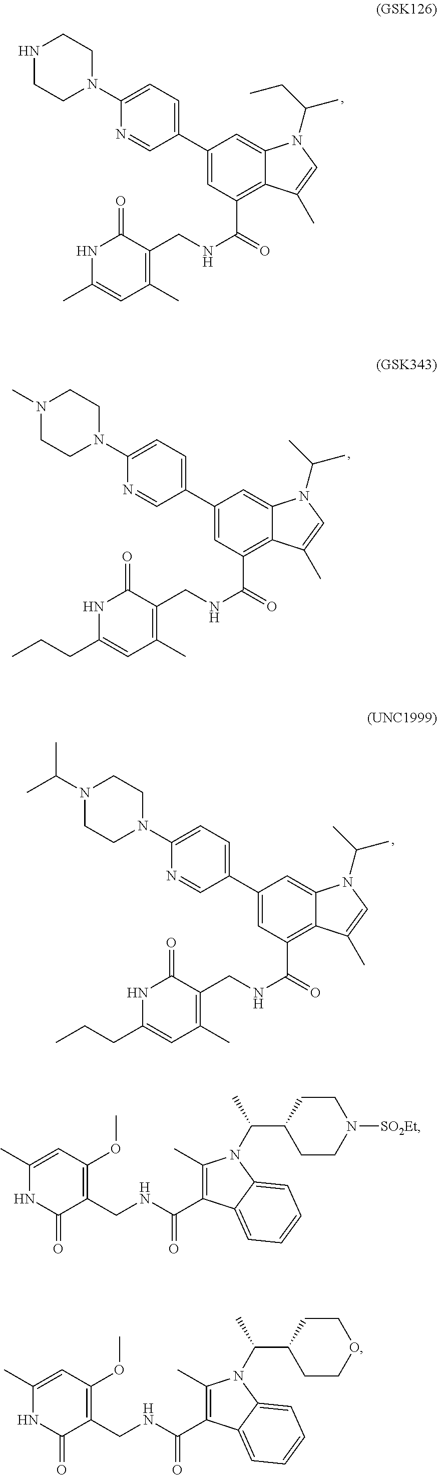

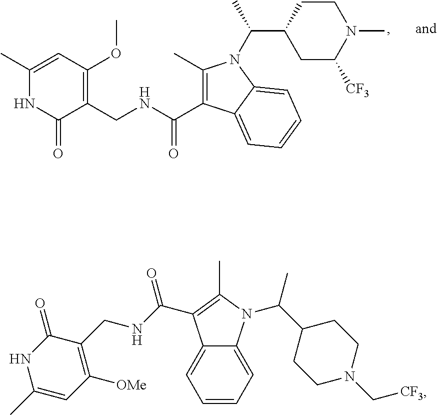

as well as those described in WO 2012/068589, WO 2013/075083, WO 2013/075084, WO 2013/078320, WO 2013/120104, WO 2014/124418, WO 2014/151142, WO 2015/023915, WO 2016/130396, and PCT/US2016/048616, the contents of each of which are incorporated herein by reference. In one aspect, the EZH2 inhibitor in the methods described herein are selected from

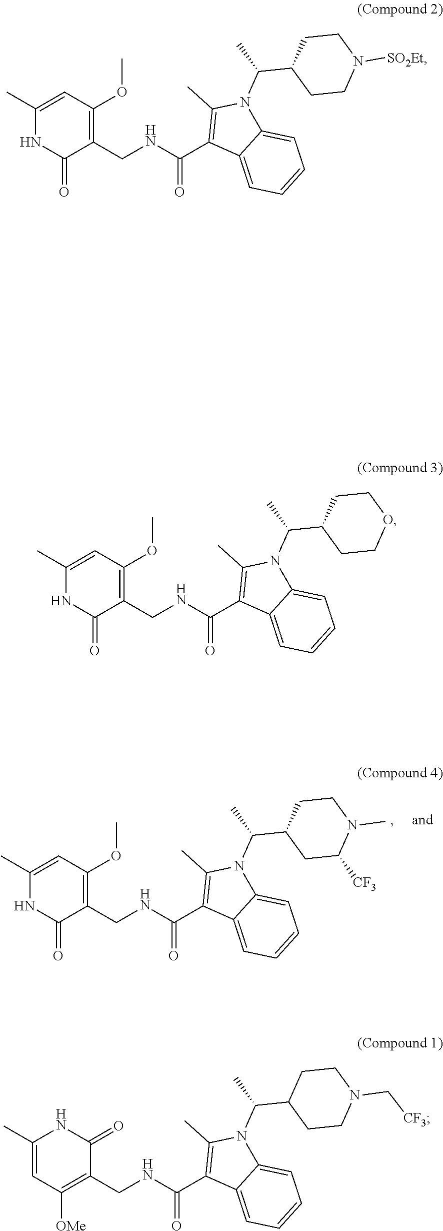

or a pharmaceutically acceptable salt thereof.

In one aspect, the initial dose of Compound 1 that is administered to a subject having cancer following the disclosed methods is from 100 mg to 1000 mg, once, twice, or three times a day. In one aspect, the initial dose of Compound 1 that is administered to a subject having cancer following the disclosed methods is from 200 mg to 1600 mg two times a day. Exemplary types of cancer include e.g., adrenal cancer, acinic cell carcinoma, acoustic neuroma, acral lentiginous melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angio sarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogenous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, primary peritoneal cancer, prostate cancer, pancreatic cancer, pharyngeal cancer, pseudomyxoma peritonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor.

In one aspect, the cancer treated by the methods or combinations described herein is selected from breast cancer, colorectal cancer, pancreatic cancer, cervical cancer, T cell lymphoma, uveal melanoma, gastric carcinoma, colorectal carcinoma, ovarian carcinoma, hepatocellular carcinoma, melanoma, and glioma. In another aspect, the cancer is selected from multiple myeloma, Hodgkin's lymphoma, non-Hodgkin's lymphoma, chronic lymphocytic leukemia, adult acute myeloid leukemia (AML), acute B lymphoblastic leukemia (B-ALL), and T-lineage acute lymphoblastic leukemia (T-ALL). In another aspect, the cancer is selected from multiple myeloma, Hodgkin's lymphoma, non-Hodgkin's lymphoma, chronic lymphocytic leukemia, adult acute myeloid leukemia (AML), squamous cell lung cancer, glioblastoma multiforme, and diffuse-type giant cell tumor. In another aspect, the cancer treated is non-Hodgkin's lymphoma. In another aspect, the cancer treated is a lymphoma such as a B-cell lymphoma.

The term “pharmaceutically acceptable carrier” refers to a non-toxic carrier, adjuvant, or vehicle that does not adversely affect the pharmacological activity of the compound with which it is formulated, and which is also safe for human use. Pharmaceutically acceptable carriers, adjuvants or vehicles that may be used in the compositions of this disclosure include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., microcrystalline cellulose, hydroxypropyl methylcellulose, lactose monohydrate, sodium lauryl sulfate, and crosscarmellose sodium), polyethylene glycol, sodium carboxymethylcellulose, polyacrylates, waxes, polyethylene-polyoxypropylene-block polymers, polyethylene glycol and wool fat.

Compositions and method of administration herein may be orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of an EZH2 inhibitor described herein in the composition will also depend upon the particular compound in the composition.

### EXEMPLIFICATION

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

### Materials and Methods

Compound 1 was prepared according to the procedures described in WO 2013/120104.

### Cell Culturing Conditions

Lymphoma cell lines were obtained from ATCC (Manassas, Va.) or DSMZ (Braunschweig, Germany) and were grown in media recommended by the vendor. All media contained 10% fetal bovine serum (FBS) and 1% penicillin/streptomycin (all media components from Life Technologies). For 4 day culturing, cells were seeded onto Compound 1-containing 96-well plates (for seeding densities see Table 1). For 10 ml cultures the seeded cell numbers shown in Table 1 were scaled up 100 times. Cell numbers were determined using the Countess cell counter (Life Technologies).

TABLE 1

| | Cell Line | Cell Number/96-well |
|---|---|---|
| 1 | Karpas-422 | 40,000 |
| 2 | OCI-LY19 | 30,000 |
| 3 | Pfeiffer | 15,000 |
| 4 | SUDHL6 | 15,000 |
| 5 | WSU-DLCL2 | 10,000 |
| 6 | SUDHL4 | 20,000 |
| 7 | HT | 20,000 |

### Isolation of Total RNA

Lymphoma cell lines were grown in 10 ml cultures (as described in 2.1). Cells were treated with 0.1% DMSO, 1.5 μM Compound 1 or in the presence of 1.5 μM other EZH2 inhibitors such as GSK126 and EPZ6438 for 4 days. After drug treatment, cells were centrifuged at 500×g for 3 min, supernatant was removed, and cell pellet was resuspended in 0.75 ml Trizol Reagent (Life Technologies, catalog #15596-026). RNA was extracted as per the manufacturer's protocol, resuspended in nuclease-free water, quantified using a NanoDrop 2000 UV-vis spectrophotometer (Thermo Scientific), and sent to Ocean Ridge Biosciences (http://www.oceanridgebio.com/) for RNA-sequencing.

For isolation of RNA from tissues: Tumor samples from a Karpas-422 mouse xenograft experiment in which mice were dosed BID with vehicle, 100 or 200 mpk Compound 1 for 4, 7, or 14 days, were obtained. Tumors were frozen in liquid nitrogen, then pulverized. A small aliquot of frozen, pulverized tissue was resuspended in 600 μl buffer RLT (Qiagen) containing 65 mM DTT, applied to a QIAShredder column (Qiagen), and centrifuged for 3 min at 20,000×g. Lysate was then transferred to a fresh microcentrifuge tube, and 600 μl 70% ethanol was added, before loading on an RNeasy column (Qiagen). RNA purification was performed as per the manufacturer's protocol, including a DNase treatment step. RNA was eluted in nuclease-free water, and concentrations were normalized following quantitation on a NanoDrop 2000 UV-vis spectrophotometer.

### Western Blotting

For western blotting, cell extracts were prepared from each of the cell lines by first separating the cytoplasmic fraction using buffer A (10 mM Tris [pH 7.9], 1.5 mM MgCl₂, 10 mM KCl, 25 mM NaCl, 0.5 mM DTT, 0.2 mM phenylmethanesulfonyl fluoride (PMSF), and protease inhibitors (Complete mini, Roche). The nuclear fraction was subsequently isolated using buffer B (25 mM HEPES, 1 M NaCl, 20% glycerol, 1.5 mM MgCl₂, 0.1 mM EDTA, 0.5 mM DTT, 0.5 mM PMSF, and protease inhibitors. Finally, the two fractions were mixed to obtain complete cell extracts of which 25 μg each were loaded per lane. SDS-PAGE was carried out using 4-12% Bis tris gels (Invitrogen). Transfer of proteins onto PVDF membrane was carried out overnight at 25 V. H3K27me3 (Cell Signaling, #9733) and total H3 (Cell Signaling, #3638) antibodies were used as 1:1000 dilutions in Tris-buffered saline with Tween (TBS/Tween). Secondary infrared (IR) dye-conjugated antibodies (Thermo) were used as 1:15,000 dilutions in TBS/Tween. An Odyssey Classic Infrared Imaging System (Li—COR) was used for signal detection.

### Lymphoma Gene Expression Profiling

Cell samples were collected and total RNA was prepared (see 2.2). RNA-sequencing from total RNA samples was carried out using the services of Ocean Ridge Biosciences, Palm Beach Gardens Fla.

RNA-sequencing data processing was carried out for the following gene expression profiling data: (1) Karpas-422 cells treated for 4 days with 1.5 μM of one of three EZH2 inhibitors: Compound 1, GSK126, and EPZ-6438. (2) 7 DLBCL cell lines treated for 4 days with GSK343 (generated in two separate experiments, with HT and SUDHL6 comprising the first experiments, the other 5 cell lines in a second experiment. For the HT and SUDHL6 cell samples in dataset 2, reads were aligned to the hg19 genome using bowtie version 0.12.9. The other datasets were aligned to the hg19 genome with Tophat 1.4.1

The aligned read files were sorted and duplicates removed using sort and rmdup functions from samtools version 0.1.182. See Li, H. et al. The Sequence Alignment/Map format and SAMtools. Bioinformatics 25, 2078-2079 (2009). Expression was estimated from the aligned reads using cufflinks version v2.1.1 (see Trapnell, C. et al. Transcript assembly and quantification by RNA-Seq reveals unannotated transcripts and isoform switching during cell differentiation. Nat Biotechnol 28, 511-515 (2010), with genome reference file Homo_Sapiens.GRCh37.73.chr.gtf downloaded from Ensembl on Sep. 12, 2013, and parameters-no-effective-length-correction-library-type fr-unstranded, and otherwise default parameters. Cufflinks fails to generate an expression estimate for a few genes; these were recorded as “NA”.

FPKM values from cufflinks' genes.fpkm_tracking output files were converted to log space by adding 1 and then taking the log base 2. Log fold change values were obtained by averaging replicates in log space, and subtracting the mean values of treated and control replicates. P-values based on t-statistics were obtained using the function mt.teststat in the multtest package from Bioconductor. See Gentleman, R. C. et al. Bioconductor: open software development for computational biology and bioinformatics. Genome Biol 5, R80 (2004); Pollard, K. S., H. N. G., Ge, Y., Taylor, S. & Dudoit, S. E. multtest: Resampling-based multiple hypothesis testing.R package version 2.16.0; and RCoreTeam “R: A Language and Environment for Statistical Computing” from http://www.Rproject.org. (2012).

Because the sample processing included enrichment for poly-A tails, further analysis was restricted to protein_coding genes. Specifically, we used Ensembl's Homo_Sapiens.GRCh37.73.chr.gtf annotation file (ftp://ftp.ensembl.org/pub/release-73/gtf/homo_sapiens/Homo_sapiens.GRCh37.73.gtf.gz), and selected the 23,083 genes annotated as having “biotype” protein_coding (22,553 genes), IG_C_gene (23), IG_D_gene (64), IG_J_gene (24), IG_V_gene (178), TR_C_gene (6), TR_D_gene (3), TR_J_gene (82), or TR_V_gene (150).

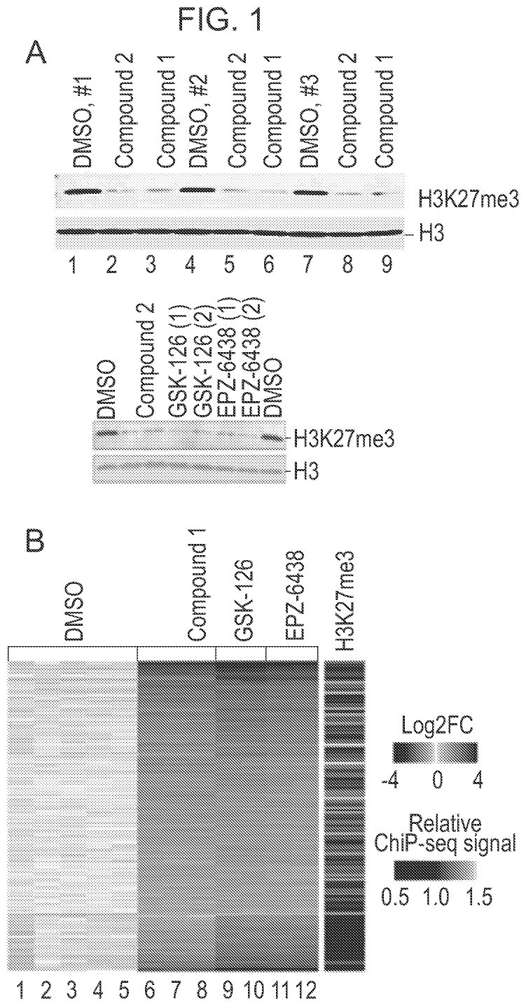

The heatmap representation shown in FIG. 1B displays expression values for the 604 genes with an absolute log fold change higher than 0.8 and a p-value less than 0.05, in all 3 treated vs control (DMSO) comparisons. Gene expression profiling for DMSO and Compound 1 treatments were carried out in triplicate (lanes 3-5 for DMSO and 6-8 for Compound 1), for DMSO, GSK126 and EPZ-6438 treatments in duplicate (lanes 1, 2 for DMSO, lanes 9, 10 for GSK126 and lanes 11, 12 for EPZ-6438). Increases and decreases in gene expression are indicated by red and blue, respectively. ChIP-sequencing was carried out in Karpas-422 cells to determine the presence of H3K27me3 across the genome. A heatmap illustrating H3K27me3 enrichment around transcriptional start sites is shown on the right. Low and high H3K27me3 enrichment are indicated by blue and yellow, respectively.

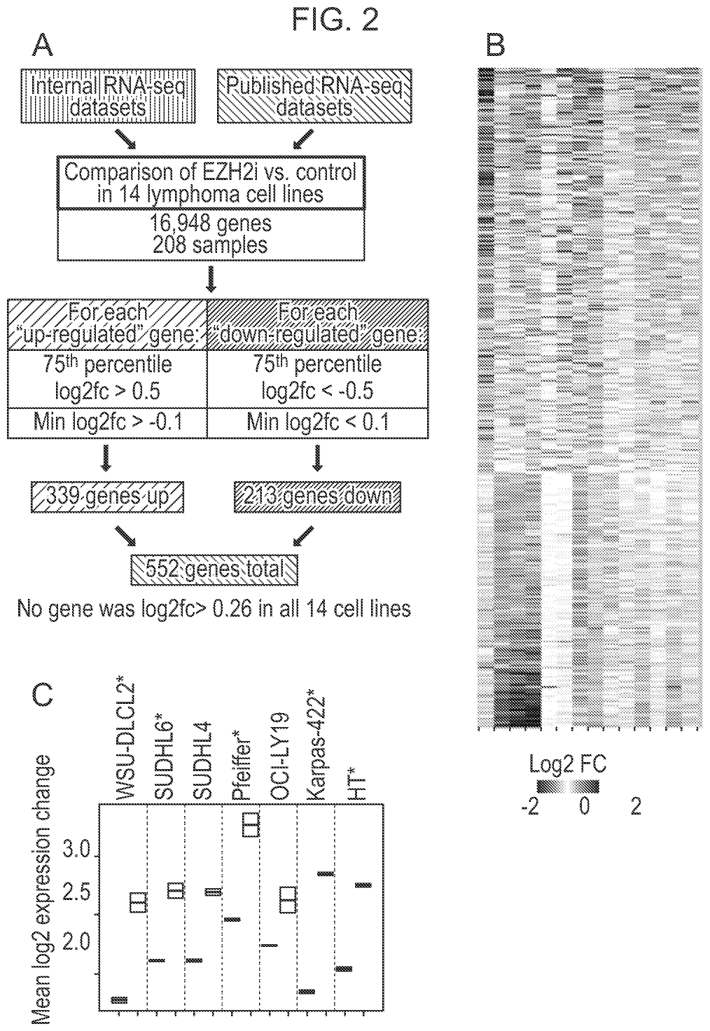

The heatmap representation shown in FIG. 2B displays expression values for 552 genes with an absolute log 2 fold change higher than 0.5 in 4 of the 14 treated vs control (DMSO) comparisons, with the gene showing no greater than a log 2 fold change of 0.1 in the opposite direction in any of the 14 comparisons. The heatmaps in FIG. 1B and FIG. 2B show the expression in log 2 space, shifted so that the mean DMSO expression value is 0.0 and shown as white. The displayed expression values then represent log 2 fold change relative to DMSO. For display purposes, the shifted expression values are capped at −4 and +4 (FIG. 1B) and at −2 and +2 (FIG. 2B), which are displayed as blue and red.

### Chromatin Immunoprecipitation and DNA Sequencing

For ChIP-sequencing, Karpas-422 cells were cultured under standard conditions. 3×10⁷ cells were treated in cell culture medium with 1% formaldehyde for 10 min. Formaldehyde-crosslinking was quenched using glycine at a final concentration of 125 mM for 10 min. Cells were washed using phosphate buffered saline (PBS, pH 7.5), pelleted and the supernatant was discarded. Cell pellets were flash frozen in liquid nitrogen. Sample processing, library generation and deep sequencing were carried out using the services of Active Motif. See website (http://www.activemotif.com/catalog/819/chip-sequencing-service) for further information.

The 50-nucleotide sequence reads were aligned to the hg19 genome using the BWA algorithm with default settings. Only reads that passed Illumina's purity filter, aligned with no more than 2 mismatches and mapped uniquely to the genome, were used in subsequent analyses. The aligned read files were sorted and duplicates removed using sort and rmdup functions from samtools version 0.1.18. See Li, H. et al. The Sequence Alignment/Map format and SAMtools. Bioinformatics 25, 2078-2079 (2009). WIG files were generated using IGVTools (version 2.2.2) count function with a window size of 25 nucleotides, an extension of 100 beyond the 50-nucleotide read length, and genome hg19. See Robinson, J. T. et al. Integrative genomics viewer. Nat Biotechnol 29, 24-26 (2011); and Thorvaldsdottir, H., Robinson, J. T. & Mesirov, J. P. Integrative Genomics Viewer (IGV): highperformance genomics data visualization and exploration. Brief Bioinform 14, 178-192 (2013). The WIG files were scaled assuming an effective genome length of 2.79B base pairs, so that the mean signal would be 1.0.

TSS locations were defined based on Ensembl genome annotations. The average signal within the interval starting at the TSS and extending 5000 nucleotides into each gene was calculated from the WIG files. The H3K27me3 TSS (0, 5000) average signal for each gene is displayed as a heatmap (FIG. 2B) using a gradation from blue (low, ≤0.5) to yellow (high, ≥1.5) via black (1.0). The signal is averaged with a sliding window of 5.

### Altered Gene Expression Upon EZH2 Inhibitor Treatment

An analysis of four independent RNA-sequencing campaigns was performed in order to identify a gene signature that correlated with inhibition of EZH2 in lymphoma, but was not necessarily a readout or predictor of response. Two internal datasets (CPI120404 and CPI130107) and two publicly available datasets (GSE40971 and GSE45982; see Beguelin, et al. Cancer Cell 2013 May 13; 23(5):677-92 and McCabe, et al. Nature 2012 Dec. 6; 492(7427):108-12) were pooled for this analysis, resulting in a combined dataset containing gene expression data for 14 distinct lymphoma cell lines treated with one of several structurally similar EZH2 small molecular inhibitors. The data set for CPI120404 was compiled from HT and SUDHL6 cell lines treated with GSK343. The data set for CPI130107 was compiled from OCILY19, Pfeiffer, SUDHL4, WSUDLCL2, Karpas-422 cell lines treated with GSK343. Gene expression profiles from the different datasets under consideration were reduced to a single probe per gene (based on the highest expressing transcript), and then combined based on gene name. This yielded a dataset with 16,948 genes and 208 samples. Biological replicates were averaged, and log 2 fold change expression values of EZH2 inhibitor treated versus control samples was made for each cell line to generate 14 individual comparisons (one per cell line).

Genes were selected that have consistent up- or down-regulation across the 14 cell lines. Specifically, for each up-regulated gene, it was required that the 75th percentile log 2 fold change for that gene across all 14 comparisons be greater than 0.5, and that the minimum log 2 fold change be greater than −0.1 (thus the gene expression change in all comparisons must be in the same direction, or deviate by only a small amount in the opposite direction). The reverse sign for these criteria was utilized for identifying down-regulated genes. Out of 16,948 genes in the combined dataset, this yielded 552 candidate genes.

No differentially expressed gene (>0.26 log 2 fold change) was common to all cell lines, therefore the signature was refined further to (1) identify the most robustly regulated genes, (2) narrow the number of genes to suit the Quantigene assay, and (3) ensure all cell lines were represented in the final signature. To achieve these goals, the 552 candidate genes were filtered further to identify genes that either (A) displayed modest changes across a majority of the cell lines analyzed, or (B) displayed a more robust change in a smaller number of cell lines. One cell line was removed from this analysis due to lack of biological replicates, and datasets from 2 additional cell lines that are less sensitive or insensitive to EZH2 inhibition in vitro were added, resulting in data from 15 cell lines being used for the generation of the final gene list. Specifically, the differentially expressed genes in list A were required to show at least 0.5 log 2 fold change in 7 out of 15 cell lines, and differentially expressed genes in list B were required to show at least 1.0 log 2 fold change in 3 out of 15 cell lines. Genes were only included in their respective list if they displayed a log 2 RPKM expression value of 2.0 or greater in at least one sample (treated or control) showing the indicated differential expression, to ensure that under some condition, gene expression was detected. List A contained 79 upregulated and 15 downregulated genes, whereas list B contained 70 upregulated and 43 downregulated genes. Only genes that appeared on both list A and B were selected for further analysis. Manual curation of expression data was performed to remove genes showing inconsistent expression changes across the individual exons of the gene (3 genes removed).

Finally, one additional gene was included from the original 552 gene list that showed some differential expression (0.05 log 2 fold change) in all 15 cell lines. 8 up-regulated genes fit this criteria, and 1 was chosen at random. Ultimately, this resulted in generation of the final gene list, which contained 37 upregulated and 9 downregulated genes Table 2. 13 out of 15 cell lines showed 0.5 log 2 fold change of at least 15 genes from the final list, and the average number of genes from the final list showing 0.5 log 2 fold change in the 15 cell lines was 27. Finally, publicly available datasets containing RNA expression data from human lymphoma tissue were analyzed to ensure the genes in the final list were detectable using standard techniques from clinical preparations.

TABLE 2

| No. | Gene Name |
|---|---|
| 1 | TRIB2 |
| 2 | TSC22D1 |
| 3 | DSTN |
| 4 | HHEX |
| 5 | S100A10 |
| 6 | GALNT10 |
| 7 | SERPINB6 |
| 8 | SPTBN2 |
| 9 | ACO1 |
| 10 | FCGRT |
| 11 | ZYX |
| 12 | MGST3 |
| 13 | ACVR1B |
| 14 | CKAP4 |
| 15 | FBXO2 |
| 16 | IFI6 |
| 17 | B9D1 |
| 18 | GNA12 |
| 19 | GIPC1 |
| 20 | PIK3R3 |
| 21 | ABCA5 |
| 22 | NPL |
| 23 | ANXA4 |
| 24 | CYTH3 |
| 25 | RHOC |
| 26 | HLA-C |
| 27 | PLEKHB1 |
| 28 | MXD4 |
| 29 | MSRB2 |
| 30 | PRKCB |
| 31 | PLCB2 |
| 32 | MT1X |
| 33 | HCP5 |
| 34 | SCD5 |
| 35 | CCDC92 |
| 36 | MPEG1 |
| 37 | ABAT |
| 38 | ACTB |
| 39 | PPIB |
| 40 | TBP |
| 41 | POLR2A |
| 42 | HAUS8 |
| 43 | CENPQ |
| 44 | RRM1 |
| 45 | ATAD2 |
| 46 | PBK |
| 47 | RAD51 |
| 48 | RAD51AP1 |
| 49 | CKS1B |
| 50 | MND1 |

In Table 2, genes 1-37 were upregulated with EZH2 ihibitor, genes 42-50 were downregulated with EZH2 ihibitor, and genes 38-41 were reference genes.

### QuantiGene® Pharmacodynamics Marker Assay Development

The gene list described in Table 2 was submitted to Affymetrix, and a multiplexed QuantiGene® assay (https://www.ebioscience.com/application/gene-expression/quantigene-plex-assay.htm) probe set on the basis was generated specific to the human genome (see Appendix B). The primer and probe sequences used to generate the multiplex assay were not made available by the vendor.

Initial tests were performed to determine the linearity of the QuantiGene® Plex Set, followed by full validation of the assay showing time- and dose-dependent gene expression changes in a mouse xenograft treated with Compound 1. Specifically, tumor samples from a Karpas-422 mouse xenograft experiment in which mice were dosed BID with vehicle, 100 or 200 mg/kg Compound 1 for 4, 7, or 14 days, were obtained (for the isolation of RNA see 2.2). For initial assay linearity testing, several concentrations of RNA (1000, 250, 62.5, or 15.625 ng final mass) were applied to individual QuantiGene® wells. The QuantiGene® assay was performed as per the manufacturer's protocol, and quantitated on a Luminex Magpix. Background mean fluorescent intensity (MFI) values for each individual gene were subtracted from MFI values generated by experimental samples. Background-subtracted MFI values were plotted against ng input RNA for each individual gene to assess linearity and signal:background ratio. Every expressed gene in the Plex Set displayed linearity at the 250 ng input level, with most genes displaying linearity even at the 1000 ng input level. However, some genes, including genes of reference, lost linearity at the 1000 ng input level. Most expressed genes exhibited a signal:background ratio of >10 at the 250 ng input level, sufficient for robust quantitation as per the manufacturer's protocol, and thus this input level was chosen for subsequent experiments.

To generate an aggregate fold change score for the overall validation experiment, gene expression changes were determined for each individual gene and combined into a single scoring metric, as defined in Gene Engagement Score Metrics.

In addition to validating the assay using a Karpas-422 mouse xenograft model, RNA from Karpas-422 cells treated with 1.5 μM Compound 1 for 4 days was also used. Samples were processed and quantitated as described above.

### Results

### Compound 1 Modulates Gene Expression Patterns in Karpas-422 GCB DLBCL Cells

In order to detect potential EZH2 inhibitor-mediated gene expression changes that could serve as a manifestation of target engagement, Karpas-422 GCB-DLBCL cells were treated with vehicle (dimethyl sulfoxide; DMSO) or Compound 1 for 4 days. Compound 1 treatment effectively reduced global H3K27me3 levels (compare DMSO treated controls in lanes 1, 4 and 7 with Compound 1-treated samples in lane 3, 6 and 9 in FIG. 1A) and caused significant changes in the expression of 604 genes (FIG. 1B). In FIG. 1A, Karpas-422 cells were treated with DMSO or 1.5 μM of various EZH2 inhibitors. The samples that were committed to RNA-sequencing were analyzed for changes in H3K27me3 levels by western blotting. Compound 2 is a predecessor compound of Compound 1 and results in similar H3K27me3 reduction. Total H3 levels were used as controls. (Bottom panel) Treatment with GSK126 and EPZ-6438 (1.5 μM) for 4 days results in a substantial loss of H3K27me3. Compound 3 is a predecessor compound of Compound 1 and results in similar H3K27me3 reduction. Total H3 levels were used as controls.

In addition, loci with H3K27me3 enrichment were determined in Karpas-422 cells by chromatin immunoprecipitation and DNA sequencing (ChIP-seq; for experimental details see 2.5). These H3K27me3 sites are indicative of PRC2 activity and often correlate with PRC2 binding sites. Consistent with EZH2's role in transcriptional repression, induction of gene expression in response to Compound 1 treatment correlated well with those genes marked by H3K27me3. In contrast, genes that were down-regulated following Compound 1 treatment were not marked with H3K27me3 (FIG. 1B). The latter group is comprised of genes that are most likely indirectly regulated by EZH2 inhibitors. These genes include cell cycle regulators promoting proliferation, similar to what has been shown previously. See McCabe, M. T. et al. EZH2 inhibition as a therapeutic strategy for lymphoma with EZH2-activating mutations. Nature (2012).

Importantly, Compound 1-mediated gene expression changes were remarkably similar to the changes observed upon treatment with other EZH2 inhibitors such as GSK126 and EPZ-6438, further supporting the conclusion that Compound 1-mediated inhibition of EZH2's catalytic activity is responsible for the observed changes in gene expression.

### EZH2-Controlled Gene Signature in GCB-DLBCL

The EZH2 inhibitor-mediated transcriptional effects observed in Karpas-422 cells prompted the question whether similar gene expression changes would be observed in other GCB-DLBCL cell models upon EZH2 inhibitor treatment. It was shown previously that EZH2 inhibitor-sensitive GCB-DLBCL cell lines were more transcriptionally responsive compared to insensitive cell lines. Moreover, the observed gene expression changes were largely different in each cell line. See McCabe, M. T. et al. EZH2 inhibition as a therapeutic strategy for lymphoma with EZH2-activating mutations. Nature (2012).

Gene expression profiling across 7 GCB-DLBCL cell lines (HT, Karpas-422, OCI-LY19, Pfeiffer, SUDHL4, SUDHL6 and WSU-DLCL2) was shown with the previously published EZH2 inhibitor GSK343. See Verma, S. K. et al. Identification of Potent, Selective, Cell-Active Inhibitors of the Histone Lysine Methyltransferase EZH2. ACS Med Chem Lett 3, 1091-1096 (2012). All cell lines were treated at a concentration of 1.5 μM for 96 hours under culture conditions that ensured optimal growth for the entire treatment period. The EZH2 inhibitor-mediated gene expression changes in each cell line were captured by RNA-seq (see 2.4 for details). While the most up-regulated genes in each cell line were indeed different, these genes tended to be up-regulated to a lesser extent in the other investigated cell lines. A number of differentially expressed genes were identified from internal RNA-seq data and published gene expression profiles using several filters (FIG. 2A). These selection criteria resulted in an EZH2 inhibitor ‘response’ gene signature that comprised 552 genes (FIG. 2B). This ‘signature’ of genes was sufficient to distinguish EZH2 inhibitor-treated from untreated GCB-DLBCL cell lines (FIG. 2C). Gene signature in 7 DLBCL cell lines is shown with the mean log 2 expression change across the up-regulated signature genes in each cell line is shown. Black: DMSO-treated. Red: EZH2-inhibitor-treated. Of note, the number of significantly altered signature genes is different for each cell line. Phenotypically sensitive cell lines (GI₅₀<1 μM in 12 day viability assays) are marked with a red asterisk.

Moreover, this gene signature was recovered in previously published lymphoma data sets, 9 which were not used in signature development. Importantly, this signature was derived from both wild type and mutant EZH2-containing cell lines, indicating that the presence of the mutant allele does not fundamentally change the EZH2-controlled gene expression program. Gene function analysis of the signature genes identified cell cycle progression and proliferation as most prominent category for the down-regulated genes. The up-regulated genes had a significant overlap with genes that are up-regulated in PC3 prostate cancer cells after knockdown of EZH2 by RNAi (see Nuytten, M. et al. The transcriptional repressor NIPP1 is an essential player in EZH2-mediated gene silencing. Oncogene 27, 1449-1460 (2008)), suggesting that the applicability of this EZH2-controlled gene signature extends beyond GCB-DLBCL. The identified gene signature (FIG. 2B) may thus be useful as a biomarker to monitor target engagement in human tumors.

### Implementation of an EZH2 Target Gene Multiplex Assay

The EZH2 inhibitor-controlled gene signature we identified in GCB-DLBCL (FIG. 2A) was pared down to 46 genes by applying a number of filters (for details see altered gene expression upon EZH2 inhibitor treatment discussed above). This gene list includes both up- and down-regulated genes. 4 genes were added to the list as references genes (genes that do not change in expression in lymphoma upon EZH2 inhibitor treatment) to result in a total of 50 genes that were interrogated (see Table 2). For each of these genes primer and probe sequences were designed, synthesized and combined in a single QuantiGene® Plex Set (see QuantiGene® pharmacodynamics marker assay discussed above).

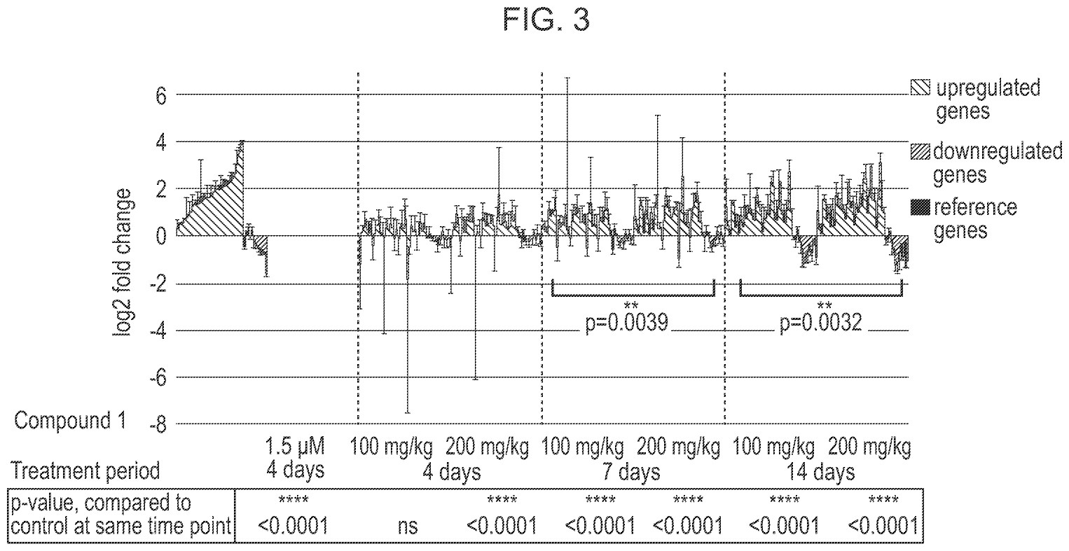

To test the fidelity of this multiplexed assay Karpas-422 cells were treated for 4 days with DMSO or Compound 1 [1.5 μM] and tumor samples from Karpas-422 xenograft-bearing animals treated for 4, 7 and 14 days with twice daily oral administration of vehicle or 100 or 200 mg/kg Compound 1 (dose and duration of treatment for each group are indicated below the graph in FIG. 3). Tumors were harvested 6 hours post last dose. Gene expression changes in the graph are ordered from left (least fold change) to right (most fold change) and grouped into upregulated (green), reference (grey) and downregulated (blue) genes.

Compound 1 treatment led to a significant increase and decrease of signature gene expression (FIG. 3, far left), indicating that the changes in gene expression reliably measure Compound 1 activity. A Karpas-422 xenograft study was carried out to determine the robustness of the gene expression signature to detect Compound 1 activity in vivo. Tumor samples from Karpas-422 xenograft bearing animals that were treated for 4, 7 and 14 days with various Compound 1 dose regiments showed significant induction of signature genes in a dose- and time-dependent manner when compared to vehicle-treated tumors (FIG. 3, 3 data sets on the right).

After 4 days of treatment with Compound 1 (100 mg/kg, BID and 200 mg/kg, BID) Karpas-422 tumors did not show the same magnitude of signature gene expression changes compared to Karpas-422 cells treated for 4 days with Compound 1 [1.5 μM] in vitro. Regardless, longer term Compound 1 treatment led to significant alteration of signature genes in Karpas-422 xenografts in vivo and supported the idea that this gene signature may serve as a readout for Compound 1 activity in clinical studies.

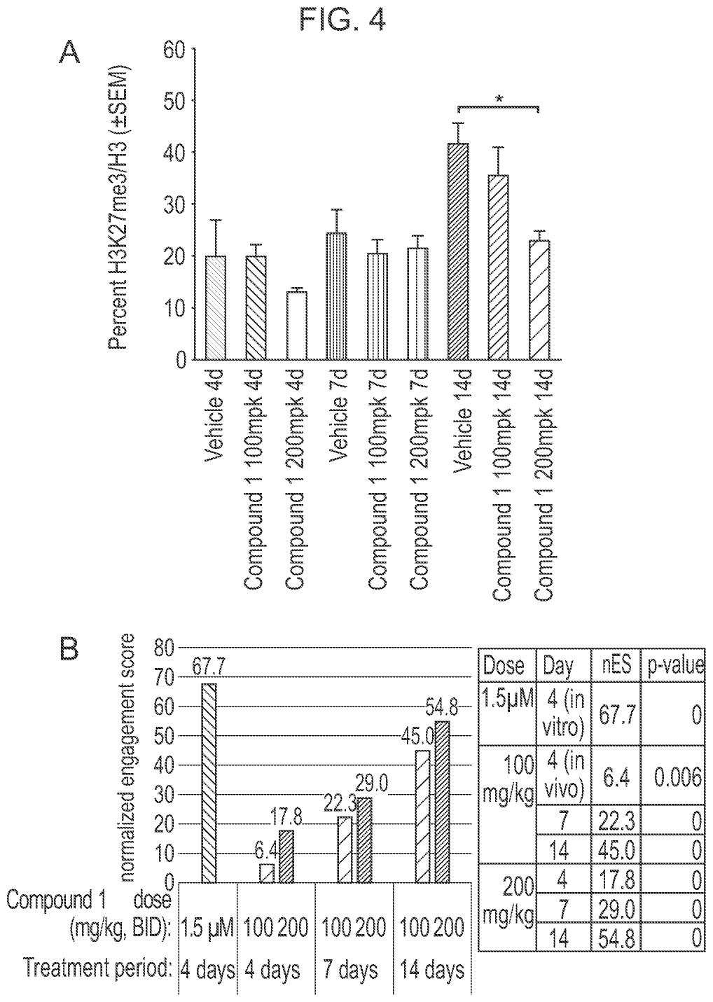

A mean signature gene expression score was calculated to compare the robustness of the Compound 1-mediated 46-signature gene expression changes with changes in global H3K27me3 levels (our primary PD marker) The H3K27me3 ELISA assay showed that a significant reduction in H3K27me3 levels (when normalized to total histone H3 levels) was only observed after 14 days of treatment (FIG. 4A). Data in FIG. 4A is represented as the mean percent of H3K27me3 normalized to total H3±SEM (t-test, *p<0.05; shows statistically significant H3K27me3 reduction between vehicle and Compound 1-treatment groups). At earlier time points the reduction was not statistically significant.

In contrast, the gene expression signature showed a robust and statistically significant change at all three time points (FIG. 3 and FIG. 4B) when vehicle arms were compared with Compound 1 treatment arms. A single gene score in FIG. 4B represents the sum of all mean fold changes in expression of each signature gene per Compound 1-treatment group compared to the respective vehicle-treatment group, calculated as descried in Gene Engagement Score Metrics. Data are represented as the aggregate gene score fold change (log 2 scale) of Compound 1-treated versus vehicle-treated tumors (light and dark blue bars). Shown is also the gene signature aggregate gene score fold change (log 2 scale) from Karpas-422 cells grown in vitro for 4 days with Compound 1 [1.5 μM] compared to DMSO-treated controls.

Thus, the ‘gene signature’ has utility as an alternative pharmacodynamics marker and may be more sensitive than global H3K27me3 in measuring Compound 1 target engagement. However, H3K27me3 remains as the most proximal and universal biomarker to measure Compound 1 activity in all proliferating cell types, while the gene expression signature is likely limited to lymphoma tumors.

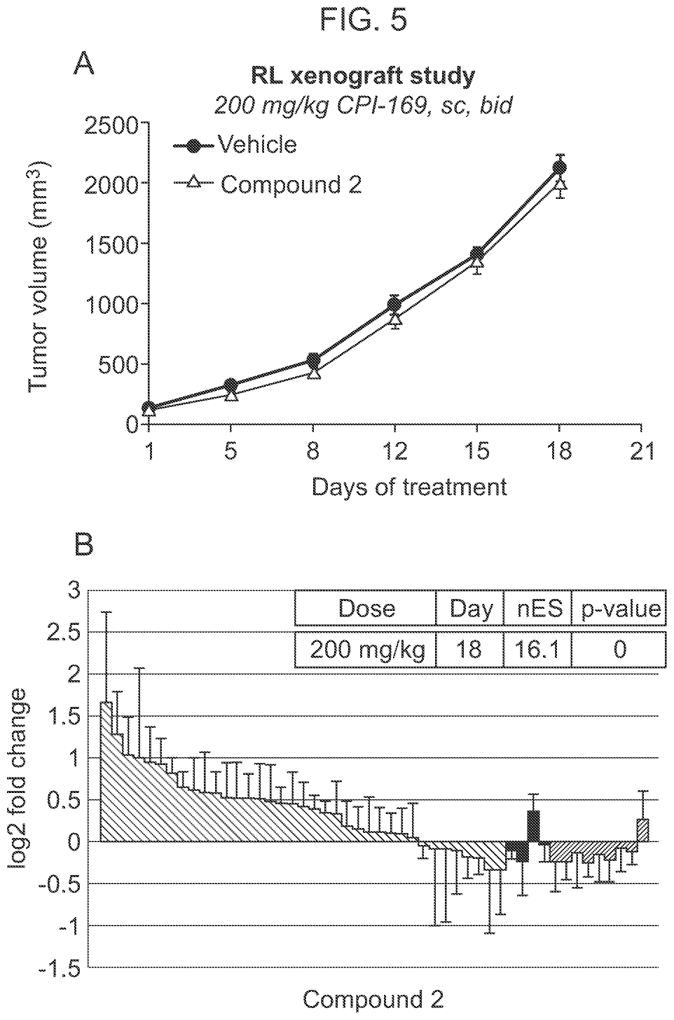

To verify the gene signature as a readout of target engagement and not phenotypic responsive, an EZH2 inhibitor-insensitive GCB-DLBCL cell line, RL, was utilized in a mouse xenograft model. The RL xenograft bearing mice were treated twice daily with 200 mg/kg Compound 2 or vehicle control for 18 days. No reduction in tumor volume was observed comparing Compound 2-treated to vehicle-treated mice (FIG. 5A). Tumors were harvested 6 hours post-last dose on day 18 of treatment and processed and analyzed using the QuantiGene® Plex assay, as described above. Despite showing a lack of efficacy, the tumors showed differential expression of several signature genes, resulting in a statistically significant gene signature score (FIG. 5B), demonstrating EZH2 target engagement.

Overall, Compound 1 significantly induced EZH2-target genes in the Karpas-422 xenograft tumors, a disease relevant, pre-clinical model of GCB-DLBCL. Moreover, EZH2 inhibitors mediated gene expression changes across a number of lymphoma cell models, including non-responsive models. This EZH2-inhibitor ‘gene signature’ was shown to have the potential to be used as a pharmacodynamics marker to measure Compound 1 activity in lymphoma. Finally, the gene signature appeared to be more sensitive compared to global H3K27me3 levels in lymphoma xenografts, further indicating its utility as a potential pharmacodynamics marker for clinical studies.

### Gene Engagement Score Metrics

The following explanation exhibits how data is treated to determine the magnitude and statistical significance of the gene engagement signature by collapsing the empirical data into a single scoring metric.

For each gene in each sample, the background median fluorescence intensity (MFI) value (recorded on the Luminex Magpix) was subtracted from the gene's measured MFI value. All resultant negative values were then set to 0, and then all background-subtracted MFI values were regularized by adding a nominal value of 1.

Following regularization, the data from each well was processed independently by first generating a normalization factor by taking the geometric mean of regularized background-subtracted MFI values for 4 housekeeping genes (ACTB, POLR2A, PPIB, TBP). The regularized background-subtracted MFI value for each gene was then divided by the geometric mean. If more than 1 technical replicate was run for an individual sample, an average value was then calculated for each gene. These values were then converted to log 2 space, and log 2 fold change values were determined by subtracting vehicle/control values from treated values.

A gene engagement score (E-Score) was developed to report a single metric representing the level of target engagement at a given dose and time point to enable pharmacodynamics relationships. The engagement score was calculated by summing the magnitude of log 2 fold change values for all predicted upregulated genes and then subtracting the sum of the magnitude of log 2 fold change values for all predicted downregulated genes. Thus, genes showing expression changes in the opposite direction than predicted reduce the engagement score. Note that log 2 fold change values less than 0.1 are reduced to 0 during consideration of the gene engagement score, since this falls below the empirically determined minimum magnitude of change required for statistical significance for this assay platform as described above.

To determine the statistical significance of the engagement score, a phenotype permutation test was performed by randomly assigning the empirically determined expression values for each gene to the control or treated condition. For each permutation, the permutation engagement score (pE-Score) was calculated similarly to the empirically determined E-Score. Random permutations were performed 1000 times. The number of instances in which the pE-Score was greater than or equal to the E-Score was determined, then divided by 1000. This number represents the p-value (or rarity) of the empirically determined E-Score. To reduce the noise in the engagement score, the mean pE-Score of all permutations was subtracted from the E-Score, thus resulting in the final reported metric, the normalized engagement score (nE-Score).

Note, that this methodology requires that if all genes change expression in the predicted direction, that the p-value will be at most 0.001 since the only permutation that can match the empirically determined E-Score is the actual empirical data. This is true even if all genes change expression by the lowest allowed value of 0.1, resulting in a E-Score of 4.6 (0.1×46). This is as equally statistically rare as a dataset in which all genes change expression in the predicted direction by a large amount (for example, 2, resulting in a E-Score of 92).

In the preceeding description, the QuantiGene® assay platform was utilized to detect changes in gene expression, and the threshold for two mean gene expression values to be statistically significant based on the technical error for this platform was empirically determined as a log 2 fold change of 0.1. Thus, any change in gene expression above log 2 fold change of 0.1 is considered statistically significant and included in all subsequent calculations and considerations. The technical error for other assay platforms utilized to detect changes in gene expression must be determined empirically. The technical error for the QuantiGene® assay platform was determined empirically by performing the assay with greater than ten samples run in technical duplicate. Expression values for each gene in each replicate were determined as described above by background subtraction, regularization, and normalization to genes of reference to correct for pipetting errors, thus accentuating the technical error associated with the platform. Mean and standard deviation values were generated for each gene in each replicate, and the coefficient of variation was determined. The median coefficient of variation across all genes for all considered assay runs was 0.0295, with only low expressed genes differing significantly from the median. Compared to the mean gene expression value for any given treatment condition 1, the mean gene expression value for any given treatment condition 2 is statistically significant at the 95% confidence interval (or 98% confidence interval) if that mean value is 2.33 times the standard deviation of the mean value for treatment condition 1. In other words, if the mean gene expression value for treatment condition 2 is greater than ((mean value of condition 1+(2.33)(mean value of condition 1)(0.0295))/mean value of condition 1, which simplifies to 1.068735, or a log 2 fold change value of 0.096, then it is statistically significant.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of treating cancer in a subject, comprising
a) administering to the subject an initial dosage amount of an EZH2 inhibitor;
b) determining the change in the expression level from a baseline level of at least five genes in the subject selected from TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1; and
c) if the change in the expression level of the at least five genes is not statistically significant relative to the baseline level of the selected genes, adjusting the initial dosage amount of the EZH2 inhibitor being administered to the subject to an adjusted dosage amount, such that the adjusted dosage amount results in a statistically significant change in the expression level relative to the baseline level of the selected genes; or
d) if the change in the expression level of the at least five genes is statistically significant relative to the baseline level of the selected genes, continuing to administer to the subject the initial dosage amount of the EZH2 inhibitor.

2. The method of claim 1, further comprising repeating steps b) and c), if necessary, until the dosage amount results in a statistically significant change in the expression level relative to the baseline level of the selected genes.

3. The method of claim 1, further comprising continuing to administer the adjusted dosage amount which results in the statistically significant change in the expression of the selected genes or greater until the treatment is terminated.

4. The method of claim 1, wherein the baseline level is determined by obtaining a biopsy from the subject's cancer prior administering the EZH2 inhibitor of and determining the expression level of the at least five genes.

5. The method of claim 1, wherein the time period between administration of the EZH2 inhibitor and determining if there is a change in expression is at least the amount of time required for the EZH2 inhibitor elicit a change in expression in at least five genes.

6. The method of claim 1, wherein the change in the expression level is of at least seven genes selected from TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1.

7. The method of claim 1, wherein the change in the expression level is of at least ten genes selected from TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1.

8. The method of claim 1, wherein the change in the expression level is of at least fifteen genes selected from TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1.

9. The method of claim 1, wherein the change in the expression level is of at least twenty genes selected from TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1.

10. The method of claim 1, wherein the change in the expression level is of at least twenty-five genes selected from TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1.

11. The method of claim 1, wherein the change in the expression level is of at least thirty genes selected from TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1.

12. The method of claim 1, wherein the change in the expression level is of at least thirty-five genes selected from TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1.

13. The method of claim 1, wherein the change in the expression level is of at least forty genes selected from TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1.

14. The method of claim 1, wherein the change in the expression level is of the genes TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, ABAT, HAUS8, CENPQ, RRM1, ATAD2, PBK, RAD51, RAD51AP1, CKS1B, and MND1 in the subject.

15. The method of claim 1, wherein the change in gene expression for TRIB2, TSC22D1, DSTN, HHEX, S100A10, GALNT10, SERPINB6, SPTBN2, ACO1, FCGRT, ZYX, MGST3, ACVR1B, CKAP4, FBXO2, IFI6, B9D1, GNA12, IPC1, PIK3R3, ABCA5, NPL, ANXA4, CYTH3, RHOC, HLA-C, PLEKHB1, MXD4, MSRB2, PRKCB, PLCB2, MT1X, HCP5, SCD5, CCDC92, MPEG1, and ABAT is upregulation, and the change in gene expression for HAUS8, CENPQ, RRM1, ATAD2, PBK, RAD51, RAD51AP1, CKS1B, and MND1 is downregulation.

16. The method of claim 1, wherein the change in gene expression or the mean change in gene expression after treatment is at least 1.96 times the standard deviation greater than the corresponding value at baseline

17. The method of claim 1, wherein the change in gene expression or the mean change in gene expression after treatment is at least 2.33 times the standard deviation greater than the corresponding value at baseline.

18. The method of claim 1, wherein the change in gene expression or the mean change in gene expression after treatment is at least 2.58 times the standard deviation greater than the corresponding value at baseline.

19. The method of claim 1, wherein the change in gene expression or the mean change in gene expression after treatment is at least 2.81 times the standard deviation greater than the corresponding value at baseline.

20. (canceled)

21. The method of claim 1, wherein the EZH2 inhibitor is 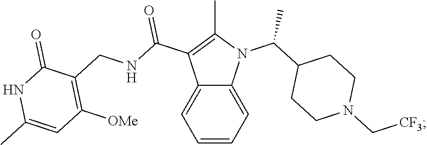 [SMILES: COc1cc(C)[nH]c(=O)c1CNC(=O)c1c(C)n([C@H](C)C2CCN(CC(F)(F)F)CC2)c2ccccc12]
or a pharmaceutically acceptable salt thereof.

22. The method of claim 1, wherein the cancer is selected from breast cancer, prostate cancer, colon cancer, renal cell carcinoma, glioblastoma multiforme cancer, bladder cancer, melanoma, bronchial cancer, lymphoma, and liver cancer.

23. The method of claim 1, wherein the cancer is a B-cell lymphoma.
